(19) United States
(12) Patent Application Publication
(10) Pub. No.: US 2012/0184047 A1
(43) Pub. Date: Jul. 19, 2012

(54) NANOPLASMONIC DEVICE

(75) Inventors: Magnus P. Jonsson, Goteborg (SE); Fredrik Hook, Goteborg (SE)

(73) Assignee: GE HEALTHCARE BIO-SCIENCES AB, UPPSALA (SE)

(21) Appl. No.: 13/498,218

(22) PCT Filed: Sep. 27, 2010
(86) PCT No.: PCT/SE2010/051034

§ 371 Date: Mar. 26, 2012

(30) Foreign Application Priority Data
Sep. 30, 2009 (SE) 0950716-1

(51) Int. Cl. G01N 21/75; G01N 1/30; B82Y 5/00

(52) U.S. Cl. 436/164; 422/82.05; 427/2.13; 977/920

(57) ABSTRACT

The present invention relates to a solution for nanoplasmonic measurement using a nanoplasmonic device with a short range order structure of trough going channels in contact with a fluid flow cell. The device is manufactured in a micro machine process comprising steps of using combined colloidal lithography, thin film deposition and etching steps on the micro/nano scale, for chemical or bio analytical sensing, and other uses. The solution makes use of shifts in the nanoplasmonic resonance, an optical property of the device that is sensitive to changes in refractive index induced by molecular reactions or other processes.

### TECHNICAL FIELD

The present invention relates to a device, use of the device, and method of manufacturing device, for nanoplasmonic application, and in particular to a sensor solution for bioanalytical sensing.

### BACKGROUND

Bioanalytical sensor devices have emerged as fundamental tools in medical diagnostics and the development of new drugs. They are also essential for both environmental monitoring and food safety. In many of these application areas it is generally not straightforward to label the target molecules prior to detection. It is therefore desirable to be able to detect target molecules directly as a change in, for example, the electrical properties of a nanowire or the mechanical properties of an oscillating quartz crystal when molecules bind specifically to receptor molecules on the sensor surface. The third main transducer principle for label-free bioanalytical sensing is based on changes in the optical properties of a surface. Surface plasmon resonance (SPR), which has been commercially available for a number of years, is today the most commonly used method. SPR is based on optical excitation of collective charge oscillations, or surface plasmons that propagate on a flat metal surface. The resonance condition for exciting surface plasmons is sensitive to changes in refractive index (RI) near the metal interface, which, in turn, can be used to monitor adsorption processes on the surface. However, to excite surface plasmons, both their energy and momentum must match that of the incoming light. For a flat metal film this means that light can only convert to plasmons at high incidence angles, which requires prism-coupling and a relatively advanced optical configuration. Alternatively, light can couple to plasmons at low angles or even at normal incidence via a grating on the metal surface. More recently it was demonstrated that periodic arrays of nanoscale holes can provide the missing momentum that is required for exciting surface plasmons and both structures have been successfully used for refractive index based plasmonic biosensing.

Thin metal films perforated with nanoholes distributed in a short-range order (no periodicity) also exhibit distinct plasmonic peaks at normal incidence. However, the decay length of the dominant plasmonic field associated with these types of structures has been shown to be around one order of magnitude lower (tens of nanometers) compared with that of periodic nanohole arrays and grating-coupled SPR (around hundred nanometers). In fact, the average penetration depth of short-range ordered nanoholes is similar to that of localized surface plasmons associated with metal nanoparticles and indeed the two systems provide very similar sensing capabilities. Although the sensitivity in peak position to changes in bulk RI is generally higher for SPR sensors based on gratings and periodic hole arrays compared with sensors based on short-range ordered nanoholes and nanoparticles, the short decay length associated with the latter two makes a thin molecular layer occupy a larger fraction of the plasmonic field. This, in turn, makes the sensitivity to changes in interfacial RI comparable. Furthermore, because a larger fraction of the plasmonic field is utilized in systems with short plasmonic fields, they also become significantly less sensitive to variations in temperature and other potentially disturbing fluctuations of the bulk solution.

It has recently been demonstrated that the short decay length of nanoplasmonic fields also may be utilized to study biomolecular structural changes on surface. This is based on plasmon shifts induced by molecules that move between differently strong regions of the plasmonic field and was exemplified by monitoring the plasmonic resonance of short-range ordered gold nanoholes coated with a thin layer of silicon dioxide (SiO₂) during the transformation of adsorbed lipid vesicles into a supported lipid bilayer. Utilizing the continuity (and corresponding electrical conductivity) of a perforated gold film, a combined nanoplasmonic and quartz crystal microbalance with dissipation monitoring (QCM-D) setup was developed, which provided two independent measures on biomolecular structural changes. It has also been shown that the nanoplasmonic field is localized to the void of short-range ordered holes, in analogy with nanoparticle plasmons being localized to particles. Using selective surface chemistry on gold and SiO₂ this was recently utilized to specifically bind biomolecular entities only to the holes, where the nanoplasmonic field is strongest. This is particularly important in situations when the total number of target molecules is low (small sample volumes and low concentrations) and can enable a large fraction of target molecules to bind to the most sensitive regions on the sensor surface.

Surface-based detection of molecules requires that the molecules come close to the sensor surface for binding to occur. Upon binding, the local concentration of target molecules at the interfacial region becomes depleted. Hence, if the actual binding reaction is sufficiently fast, the rate of binding will be determined by diffusion across a growing depletion zone. The extension of the depletion zone can be efficiently reduced by flowing the target solution parallel to the sensor surface, resulting in an increased binding rate.

### SUMMARY

It is therefore an object of the present invention to provide solutions for a nanofluidic network comprising parallel fluidic channels in two dimensions (2D) where a region or regions of the inner part of the channels are sensitive to changes in refractive index (RI) induced by biomolecular adsorption events or other processes. The invention may be used for flow-through chemical, biomolecular or gas sensing or other types of sensing. The structure may also be used as a nanofilter with or without the combination with sensing.

The present invention relates to short-range ordered nanoplasmonic pores. This structure has the benefit of being continuous and enabling liquid access to both sides of the pores. At the same time, the short-range order makes the sensitivity, at least partly, localized to the inside of the pores where the analyte will flow. With respect to the development of applications, such as tools for diagnostics and drug screening it is essential to be able to use low-cost and scalable nanofabrication techniques. Therefore, a parallel fabrication scheme is presented. Not only the nanoholes are made all at the same time, but the method also enables around 50 devices/samples to be fabricated in parallel on one single wafer. The number of devices per wafer may be further increased by decreasing the size of each device and using larger wafer.

Since the nanochannels/nanopores are organized in a parallel fashion (in two dimensions) the solution has the potential for high through-put.

This is provided in a number of aspects of the present invention, wherein a first is a nanoplasmonic device comprising a membrane with at least one layer of conducting material, wherein the membrane is perforated with a plurality of through going channels, and wherein the relative position of the channels are arranged so as to form a pattern with no long-range order.

The spatial centre-to-centre length between nearest neighbour through going channels may be of the order of 1 to 10 000 nanometres, preferably of the order 10 to 1 000 nanometers, and more preferably of the order 50 to 500 nanometers. The through going channels may have a diameter of the order 10 to 500 nanometers preferably of the order 25 to 250 nanometers, and more preferably 50 to 150 nanometers. The electrically conducting layer comprises at least one of gold, silver, palladium, and platinum. In the device, a thickness of the membrane may be of the order of 1 to 1 000 nanometers, preferably 5 to 500 nanometers, and more preferably 10 to 100 nanometers. The electrically conducting layer may also comprise at least one of chrome, titanium, chrome oxide, titanium oxide, and tantalum oxide.

The membrane may further comprise at least one mechanically stabilizing layer. The mechanically stabilizing layer may be one of an insulating layer or semi conducting layer.

Another aspect of the present invention is provided, a measurement system for measuring molecular reactions, comprising:
at least one nanoplasmonic device according to the first aspect of the invention;
a fluid flow cell arranged so as to provide contact by fluid in the fluid flow cell with the nanoplasmonic device;
a system for determining optical properties of the nanoplasmonic device;
a control and analysis system (302) in electrical connection with the system for determining optical properties.

Yet another aspect of the present invention is provided, a method of manufacturing a nanoplasmonic device, comprising the steps of:
forming a membrane;
forming a plurality of through going channels in the membrane and wherein the relative position of the channels are arranged so as to form a pattern with no long-range order.

The steps of forming the membrane and the channels may comprise the steps of:
depositing colloids on a mechanically stabilizing layer with a spatial length in a range of 1 colloid per 1 to 10 000 nm;
evaporating an electrically conducting layer on the mechanically stabilizing layer and the colloids;
removing the colloids forming holes in the electrically conducting layer;
coating the mechanically stabilizing layer and conducting layer with an insulating layer;
defining and removing a window structured portion in a substrate back side and removing a windowed structure portion of the mechanically stabilizing layer exposed after removal of the windowed structured portion of the substrate;
producing through going channels through the mechanically stabilizing layer and the conduction layer forming nano sized pores with nanoplasmonic properties.

The step of depositing colloids may comprise depositing the colloids in a homogenous short-range order. The method may further comprise an initial step of depositing an insulating or semi conducting layer on the substrate.

The mechanically stabilizing layer may be one of a substrate layer or a separate insulating or semi conducting layer.

Still another aspect of the present invention is provided, a method of measuring molecular reactions with a nanoplasmonic device, comprising the steps of:
placing a nanoplasmonic device with no long-range ordered through going channels in contact with a fluid flow cell;
providing a reactant to a fluid;
providing the fluid with the reactant to the fluid flow cell;
determining optical properties of the nanoplasmonic device over time;
relating changes of the optical properties to molecular reactions.

Another aspect of the present invention is provided, a sensor consumable comprising a nanoplasmonic device according to the first aspect and further comprising a holding structure arranged to be held by a measurement system.

These and other aspects of the invention will be apparent from and elucidated with reference to the embodiments described hereinafter.

### BRIEF DESCRIPTION OF THE DRAWINGS

In the following the invention will be described in a non-limiting way and in more detail with reference to exemplary embodiments illustrated in the enclosed drawings, in which:

FIG. 1 illustrates schematically a manufacturing process of a device and the device according to the present invention;

FIG. 2 illustrates schematically in a block diagram a system according to the present invention;

FIG. 3 illustrates schematically in a block diagram a sensor system according to the present invention;

FIG. 4 illustrates schematically a method according to the present invention.

### DETAILED DESCRIPTION OF PREFERRED EMBODIMENTS

The present invention comprises a micro machined nanoplasmonic device and method for manufacturing this device using combined colloidal lithography, thin film deposition and etching steps on the micro/nano scale, for chemical or bio analytical sensing, and other uses. The solution makes use of shifts in the nanoplasmonic resonance, an optical property of the device that is sensitive to changes in RI induced by molecular reactions or other processes. The molecular reactions may be controlled by changing the composition of provided analytes and/or physical configuration of the device. The device may be used as a sensor consumable in a measurement system and this will be discussed in more detail later in this document.

The nanoplasmonic device comprises a membrane with through going channels formed in a pattern without any long range order. The pattern comprises for instance a completely random distribution of the channels but also structures where there is only a short-range order. Short-range order may be defined as regularity in the arrangement of the channels such that the centre-to-centre distance distribution of nearest neighboring channels is narrower than the centre-to-centre distance distribution of nearest neighboring channels for completely randomly distributed holes. A system with long-range order is defined as a substrate with channels arranged such that there is a periodicity in the centre-to-centre distance-distribution of channels in at least one direction. It should be noted that the channels in the present invention may be distributed in so called quasi-order, e.g. structures that are defined by a particular pattern, but still lack long-range order. In the present invention the channels may be distributed so as to not exhibit a long-range order larger than 3, preferably 10, or more preferably 100 average nearest neighbor distances. Nearest neighbor distance may be defined as the distance between channel center to center distances.

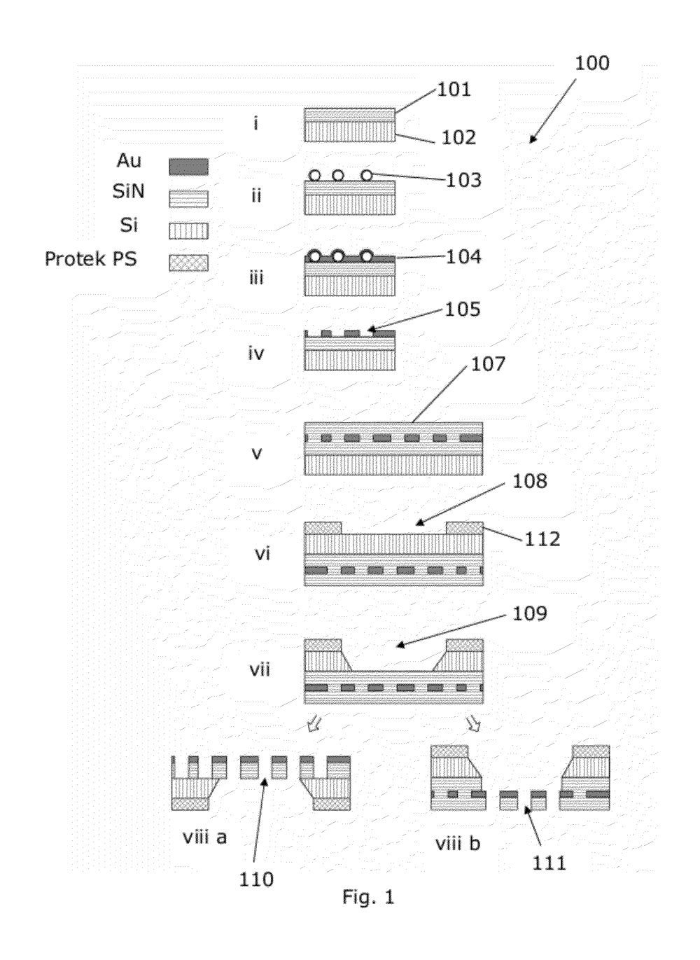

The fabrication scheme of the device is presented in FIG. 1 and is described below. In brief, as an example, a 65 nm thick gold film 104 perforated with 150 nm in diameter short-range ordered nanoholes 105 is fabricated on a SiN 101 coated (200 nm) Si wafer 102 using colloidal lithography (steps i-iv). Potentially, other materials, such as silicon dioxide (SiO₂) may be used instead of SiN depending on the application. The SiN layer acts as a mechanically stabilizing layer and is preferably insulating; however a semi conducting material may be used as well. It is possible to fabricate the device without this type of mechanically stabilizing layer and rely on the Si substrate it self for this; however, the device will be less rigid in this case which for many applications is of no importance. Colloidal lithography is chosen both because it may provide a short-range order and also because it is a simple, fast and low-cost method to produce nanostructures on large areas. The nanoholes are subsequently coated by an additional 200 nm SiN layer (step v) 107. This layer will protect the nanohole structure during wet etching. As described below, either this or the first SiN film will increase the mechanical stability of the final structure. Open squares 108, circles, or some other shape in a negative photoresist (ProTEK PSB-23, Brewer Science, UK) are subsequently defined on the backside of the wafer by conventional UV-lithography (step vi). Around the squares, thin slits are made to define the dimensions of each final sample. All open areas may then be removed by Si wet etching in tetra-methylammonium-hydroxide TMAH, while areas protected by the resist are not attacked (step vii). As an alternative, a patterned SiN (or silicon dioxide SiO₂ or similar materials) layer on the backside may be used as mask during wet etching of Si. Other etch solutions then TMAH, such as potassium hydroxide (KOH), may also be used for Si etching. With proper masking materials, dry etching of Si may also be used. The metal nanohole structure on the front side of the wafer is protected by the top SiN coating. The Si in the open squares is completely removed after around 13 hours in TMAH and the first deposited SiN film acts as etch stop. This will result in squares 109 of thin free-hanging SiN/metal nanohole/SiN membranes supported by the Si wafer outside the membranes. Due to etching also in the slits, the wafer may be easily divided into approximately 50 samples with normal tools, e.g. tweezers. The final step is to open up the nanoholes into through-going plasmonic channels or pores, for which reactive ion etching (RIE) was chosen. This may done either from the front side (step viii a) or the backside (step viii b) of the wafer. The main difference between these approaches is that the latter opens the channels only in the membrane area and hence, the gold only becomes accessible in this region. For both approaches the gold film itself was used as etch mask during RIE. It should be noted that more or less samples may be arranged on the wafer depending on size of wafer and/or size of sample—any number of samples between 1 to 500 for instance.

The successful fabrication of nanopores may be verified by, for example, scanning electron microscopy and the plasmonic properties of the samples may be investigated by micro extinction spectroscopy in an ordinary microscope equipped with a back-thinned 2D-CCD spectrometer.

It is important to note that the gold itself was used as etch mask during RIE. The reason for this was primarily to avoid alloy-formation between the gold and an additional masking layer (e.g. chrome) during RIE. However, materials other than gold or multilayers of different materials may also be produced with the fabrication method. The parameters for the RIE process were optimized for high selectivity between SiN and gold (approximately 19:1), with etch rates of around 230 nm/min and 12 nm/min, respectively. With a ≈130 nm thick SiN top protection layer the gold is exposed to the RIE for around 116 s, which etches the gold around 23 nm.

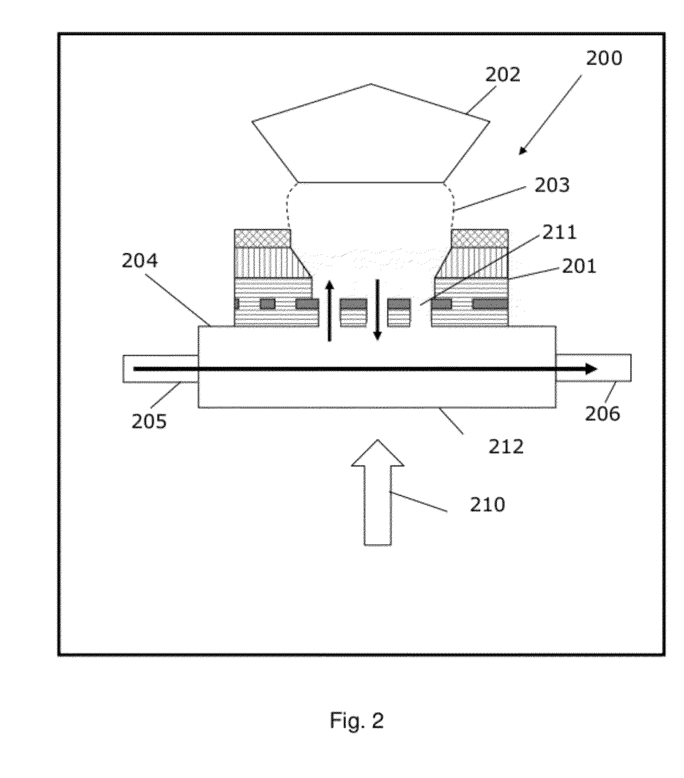

The plasmon resonance is sensitive to changes in refractive index (RI). This may be verified by flowing liquid through the nanopores, which induces a bulk RI shift of approximately 0.33 for water. The samples may be treated in an Ultra Violet (UV) ozone chamber to make them more hydrophilic. It should be noted that in measurements an air immersion microscope objective with low numerical aperture may be used to minimize the fraction of scattered light that is collected by the objective; however, other optical configurations may be used. FIG. 2 shows a measurement setup 200 with an optical detector 202, e.g. comprising a lens, optionally an optical excitation device, and a CCD, detecting optical properties of a sample located with the nanoplasmonic device 201. A fluid flow cell 204 with a fluid inlet 205 and outlet 206 is located in contact with the nano channels 211. The fluid cell is made of a light permeable material 212 at least partly. Fluid from the fluid flow cell is allowed to interact with the nano channels 211. Between the nano channels and the optical detector device an optional droplet 203 of suitable fluid, e.g. water or ethanol, may be located as a second liquid reservoir depending on the application. The concept may also be used in combination with fluidic channels on one or both sides of the device. In one embodiment, an optical source 210 is provided on the opposite side of the fluid flow cell as compared to the optical detector, thus detecting light in an absorption mode. The optical source may for instance provide a collimated beam of white light, or light in a specific wavelength depending on application.

The plasmonic resonance peak can be measured, for example, using micro extinction spectroscopy or by dark-field spectroscopy. Shifts in the plasmonic resonance may be measured as changes in the peak position itself or using peak tracking algorithms, such as the centroid method. Other shifts can also potentially be used, such as changes in amplitude or other parameters. The plasmonic resonances may also be detected using a reflective technique, where the light source is located at the same side as the detector and reflected light from the nanopores are measured.

The nanoplasmonic channels may be used for real-time monitoring of specific biomolecular recognition reactions. In order to achieve high signal-to-noise ratio with high temporal resolution several factors may be considered. Thin gold films (≈42-45 nm after RIE) may be chosen instead of opaque metal films. These thin films transmit a significant fraction of light in the visible (also without the holes) and the plasmon resonances often appears as peaks and not as dips in the extinction spectrum. This is in contrast to the phenomenon of enhanced transmission in thick perforated metal films for which dips are observed in the extinction spectrum. For measurements on the micrometer scale in particular it is of high importance to maximize the amount of transmitted light in order to utilize the full dynamic range of the detector also at low integration times, e.g. 14 ms. This, in turn, is advantageous for maximizing the signal-to-noise ratio, which ultimately determines the lowest concentration that may be detected with the plasmonic sensor. For the same reason, an ultrasensitive back-thinned 2D CCD spectrometer with high dynamic range may be used. Further, the centroid (center of mass) of the plasmon peak may be monitored instead of the peak position itself, which has shown to improve the signal-to-noise ratio significantly for sensing methods based on peak tracking.

A possible scheme for flow-through measurements is presented: The sample is placed in a flow cell with the gold side facing down into the flow cell. The liquid compartment on the other side may be a drop of buffer in contact with a water immersion microscope objective (e.g. with 63 times magnification, 63×). With this configuration it is possible to functionalize and perform rinsing steps in the flow cell before flow-through measurements and without needing to exchange the liquid in the upper liquid compartment. The target molecules may then subsequently be added to the buffer droplet using a syringe or similar to allow flow through measurements. It is possible to bind target molecules as they flow through the nanoplasmonic channels. One motivation with flow-through sensing is to increase binding rates. It is therefore of high importance to maintain a high temporal resolution when optimizing the signal-to-noise ratio.

The region inside the channels close to the gold will to some extent be located within a nanoplasmonic field. This has been exemplified for similar, but non-through going nanoplasmonic wells for which shifts in the plasmon resonance could be used to monitor adsorption of biomolecules specifically to glass regions in the bottom of those wells. Nonspecific adsorption of NeutrAvidin to the SiN inside the pores close to the gold is therefore expected to induce a shift in the plasmon resonance. It is therefore advantageous to control the surface chemistry selectively for gold and SiN in order to enable measurements of only the response induced by specific adsorption of, for example, NeutrAvidin to biotinylated gold. This may, for example, be achieved by functionalizing the gold with thiol-PEG:thiol-PEG biotin (for instance 1:1) on gold and subsequent passivation of the SiN with PLL-PEG. PLL-PEG is known to provide highly protein resistant layers on SiO2, while not adsorbing to thiol-PEG and has been shown to successfully prevent protein adsorption also on SiN.

In conclusion, in the present invention a scheme for parallel fabrication of arrays of nanoplasmonic trough going channels is provided, where the number of samples produced simultaneously is limited only by the size of the wafer and samples. The channels are distributed without any long-range order, mainly to localize the sensitivity to within the channels where molecules may be rapidly exchanged. Optionally the channels are arranged in a short-range order. The fact that biomolecular recognition reactions may be monitored accurately and at high temporal resolution shows the potential of the structure for flow-through biosensing. The integration with microfluidics on both sides of the nano channels may help to utilize the benefits of flow-through sensing. It should also be noted that an array of nanoplasmonic channels with liquid access to both sides may be an interesting platform for pore-spanning artificial cell membranes, which possibly may be used to monitor single membrane ion channels. The nanoplasmonic component of the structure presented in this invention adds the possibility to measure transport of molecules through such artificial membranes not only by electrical means, but also optically. In contrast to existing techniques, transport of both charged and non-charged molecules may then be measured. It should be noted that the through going channels need not be shaped cylindrically, but may be conical, hour glass, or irregularly shaped. Furthermore, the channels need not be shaped as circles in the surface plane but may take other shapes as well, such as ellipsoidal or even polygonal. The geometrical properties of the channels may be used for fine tuning the nanoplasmonic properties.

The spatial centre-to-centre length between nearest neighbour through going channels may be of the order of 1 to 10 000 nanometres, preferably of the order 10 to 1 000 nanometers, and more preferably of the order 50 to 500 nanometers. The through going channels may have a diameter of the order 10 to 500 nanometers preferably of the order 25 to 250 nanometers, and more preferably 50 to 150 nanometers. The electrically conducting layer comprises at least one of gold, silver, palladium, and platinum. Furthermore, other metals may be used for forming the electrically conducting layer; in fact, even some semi conducting materials may be used, such as for instance gallium phosphide. In the device, a thickness of the membrane may be of the order of 1 to 1 000 nanometers, preferably 5 to 500 nanometers, and more preferably 10 to 100 nanometers.

Further use of the nanoplasmonic channels is to act as nano sized filters with integrated nanoplasmonic sensing. This may, for example, enable selective measurements of only a fraction of molecules that are sufficiently small to be able to flow through the channels. For example, this may significantly simplify the preparation in blood analysis.

The fabrication of the nanoplasmonic devices using one possible method will now be described in more detail with reference to FIG. 1. It should be understood that this is only an exemplary embodiment and that some of the steps may be changed to other steps, the order of the steps interchanged, and that the dimensions and ratios mentioned is also exemplary.

First, an approximately 200 nm thin silicon nitride (SiN) film 101 is deposited (i) on a silicon wafer 102 (e.g. 275 μm thick, 2 inches in diameter) using plasma-enhanced chemical vapor deposition (PECVD). A radio frequency (RF) signal may be applied during PECVD and may be alternated periodically to minimize the stress in the SiN film. Alternatively, other types of SiN, a silicon dioxide film or even multilayers may be used instead of the SiN film described above. Furthermore, the SiN film and Si substrate may take various thicknesses, e.g. ranging from 1 nm to 1 micrometer for the SiN film.

Short-range ordered gold nanoholes is subsequently fabricated on the SiN film using colloidal lithography. First, a 5 w % aluminium chloride hydroxide is added to the SiN layer for 60 s, followed by rinsing in water and drying with nitrogen. This renders the SiN surface positively charged. Next, 0.1 w % 150 nm in diameter negatively charged polystyrene colloids 103 may be added (ii). A balance between the electrostatic interaction between the surface and the colloids and the repellence between the colloids make them arrange into a homogeneous short-range order on the surface. The wafer is then rinsed in water, followed by fuming ethylene glycol, water again and finally blow-dried with nitrogen. The ethylene glycol helps to minimize particle movement and agglomeration during the drying process. Mild oxygen plasma etch may then be performed to remove the electrolyte from the SiN film, but without removing the colloids. This step has shown to enable a better adhesion of metals to the underlying film. It should be mentioned that the many parameters in the recipe for the colloidal lithography may be varied. For example, multilayers may be used instead of a single ACH layer or the colloids may be shrunken before metal deposition, for example, using oxygen plasma treatment. Next, e-beam assisted evaporation of 1 nm chrome, 65 nm of gold 104 and 1 nm chrome is deposited (iii) onto the sample, where the chrome serves as adhesion layers. The chrome layers are used for improved adhesion to neighboring materials. It should be appreciated that the thickness of the gold layer is not limited to the exemplified 65 nm but may range from 1 to 500 nm (depending on colloid diameter) and that the gold may be replaced with other materials, such as silver, palladium or platinum, mixtures thereof, or multilayers of two or more materials. Also the chrome layer may be different, e.g. from 0.1 nm to 10 nm when used as adhesion improver. Furthermore, chrome may be replaced with at least one of titanium, chrome oxide, titanium oxide and tantalum oxide. Removing (iv) the colloids by tape stripping or other means then creates metal nanoholes 105 on the surface. Next, the nanohole structure is coated (v) by around 200 nm SiN 107 using PECVD. The nanohole structure is now arranged in a non-ordered fashion when viewed on a larger scale, i.e. in the order of micrometers or larger.

UV-lithography (vi) may then be performed on the backside of the wafer to define open squares or other open areas 108 in negative photo resist ProTEK PSB-23 112. Thin slits (not shown) may also be produced in this step to define the dimensions of the final samples. First, a ProTEK PS Primer is spin coated onto the backside of the wafer at 1000 rpm (revolutions per minute) for 60 s and baked at 110° C. for 60 s and at 220° C. for 120 s (all baking steps may be done on hot plates or an oven). The photoresist ProTEK PSB-23 is then spin coated at 3000 rpm for 60 s and baked at 110° C. for 60 s. The inverse pattern of a chrome mask is then transferred to the resist in a mask aligner, e.g. Karl Süss MJB3-UV400 by exposure three times for 30 s at around 5 mW/cm². After another baking at 110° C. for 120 s the resist may be developed in Ethyl Lactate for around 2 min followed by rinsing in isopropyl alcohol (IPA) and blow dried with nitrogen. The final step for UV lithography is baking at 220° C. for 120 s. Patterned SiN or SiO2 may be made by, for example, photolithography and etching steps and used instead of the ProTEK PSB-23.

The wafer is then immersed (vii) in tetra-methyl-ammonium-hydroxide TMAH, which does not attack ProTEK PSB-23, but etches Si anisotropically. The SiN film on the front side of the wafer protects the metal nanostructure from being attacked by the etch solution. The Si in the open squares is completely removed after around 12 hours in TMAH and the lower SiN film acts as etch stop. The wafer is then thoroughly rinsed in water and blow-dried with nitrogen. This results in squares 109 of thin free-hanging SiN/metal/SiN films with the nanoholes in the metal film and with the Si wafer as support outside the squares. Other etching techniques than TMAH may also be used, such as potassium hydroxide wet etch and even dry etching methods if other masking layers are used. The Si may also be etched in slits defined in the UV-lithography step, which enables the wafer to be easily divided into approximately 50 samples depending on size on devices and wafer. The final step was to open up the nanoholes into through-going plasmonic channels using reactive ion etching (RIE). This is done either from the front side (viii a) or the backside (viii b) of the wafer, where the latter approach provide nano pores 110 with the gold accessible in the window area and the former provide nano pores 111 with no gold accessible in the window area. For both approaches the gold film itself acted as etch mask during RIE (see FIG. 1 step viii a and b). The RIE may be performed for varying times using NF₃ at a flow rate of 50 sccm, at a power of 70 W and a pressure of 10 mTorr. Other RIE recipes may be used, for example including the gas CF₄.

The plasmonic devices may be quality analyzed, for instance taking extinction spectra of the devices using a conventional microscope equipped with a 100 W quartz tungsten halogen light source and a back-thinned 2D CCD spectrometer, e.g. QE65000 from OceanOptics Inc. (trademark). The spectrometer may be controlled with a custom designed program, for instance using LabView program from National Instruments Inc. (trademark). First, a dark spectrum may be taken without illuminating the spectrometer. This may be followed by recording a reference spectrum and finally the device is placed in the light path and the extinction spectrum may be acquired and displayed according to

$$E ( λ ) = 1 - \frac{{I}_{sample} ( λ ) - {I}_{dark} ( λ )}{{I}_{reference} ( λ ) - {I}_{dark} ( λ )}$$

with extinction values from 0 to 1. The optical properties may also be displayed using other relations.

For sensing experiments a water immersion objective with 63 times magnification may be used, where the immersion droplet is used as one of the two liquid compartments on each side of the nanoplasmonic pores. A reference spectrum may be taken before measurements using the same objective and a droplet on a microscope slide. The fact that the absolute values of intensities and peak position might not be absolutely correct is not critical, because we are only interested in shifts in the plasmon resonance for these experiments. In biosensing experiments, the spectrum is fitted to a polynomial and the centroid (centre of mass) of the peak may be calculated and plotted using a custom designed LabView program.

Negative control for plasmonic characterization may be performed. For samples with nanoholes on a flat thin SiN membranes (no holes through the SiN) may be fabricated as above, but without the SiN top coating. Instead, a polymer, e.g. ProTEK B3 from Brewer Science may be used for protection during wet etching in TMAH. This polymer may subsequently be removed in methyl isoamyl ketone (MIAK). Because no liquid may go through these nanoholes, such samples may be used to investigate if plasmon shifts may be observed also for changes in RI on the SiN side of the sample. No shift is observed when a drop of ethanol is placed on the backside of the sample, but the plasmon resonance is only shifted when the drop is placed on the side where the gold is exposed.

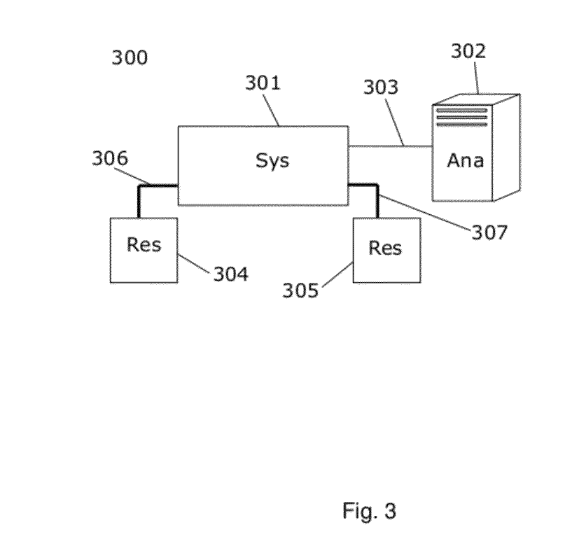

FIG. 3 illustrates a measurement system 300 according to the present invention with a measurement setup 301 as described in FIG. 2 suitably encased, a fluid reservoir 304, fluid waste reservoir 305, and measurement control unit 302. The reservoirs are connected to the measurement setup with suitable tubing 306 and 307. The fluid flow may also be controlled by fluidic channels on both sides (not shown) of the device. The measurement control is connected to the measurement setup with suitable parallel or serial communication and control interface 303, e.g. Ethernet, GPIB HPIB, VXI, I2C, RS232, and so on. The measurement system may be combined into one single casing with appropriate user interfaces, such as ports for filling or emptying the reservoirs, changing the nanoplasmonic device, adding droplets of fluid at the optical detection side of the nanoplasmonic device, cleaning and so forth. The system has a nanoplasmonic device receiving unit (not shown) that receives the nanoplasmonic device and holds the nanoplasmonic device during measurement. This receiving unit is arranged to provide easy changing of nanoplasmonic devices while at the same time providing secure holding: e.g. the receiving unit comprise a structure to which the nanoplasmonic device fits into tightly and with some clamping means for holding the structure still—i.e. with some quick release functionality. The structure may comprise a recessed portion or a slot for sliding in the nanoplasmonic device sideways into the slot. The clamping means may be for instance some kind of spring solution or frictional solution. However, it should be appreciated that the receiving unit need not be arranged with a receiving structure, but that the nanoplasmonic device is only held by clamping means, for instance as a microscope glass slides are held by one or more spring like clamps during operation in an optical microscope. In one embodiment the nanoplasmonic device is glued to a separate holding structure, for instance made of metal, and this holding structure is in turn fastened in the measurement setup by clamping, frictional solution, or other means. Advantageously, the nanoplasmonic device, with or without separate holding structure is mounted in a leak sealed manner, for instance by using suitable o-rings or similar sealing means.

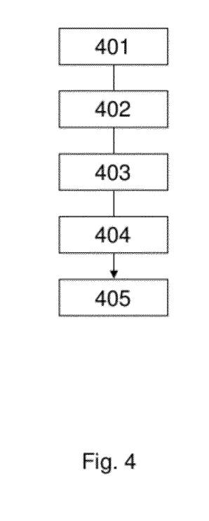

A measurement method may take the following form with respect to FIG. 4:
401. Placing a nanoplasmonic device according to the present invention in the measurement setup 301. The nanoplasmonic device may be arranged locally or purchased with pre arranged affinity for certain molecular reactions depending on type of measurement to be performed.
402. Providing fluid comprising molecules of interest at one side of the nanoplasmonic device. A fluid with appropriate molecular composition is provided in the reservoir or directly into the fluid cell chamber.
403. Detecting optical properties of nanoplasmonic device. A CCD detector or similar is used for determining the optical properties at the interaction volume, i.e. in the nano pores.
404. Measuring changes of the optical properties over time in relation to a molecular reaction process at the nanoplasmonic device. For many types of measurement it is of interest to detect the variation of a reaction over time or for increasing the signal to noise ratio by integrating over time.
405. Analyzing and presenting the measured changes. Depending on type of measurement different types of analysis may be of interest for determining peak or dip positions, average levels, derivative or integrated effects and so on.

The nanoplasmonic system may be integrated into existing microscope platforms or developed and sold as stand alone instruments. The nanoplasmonic device may be sold as consumable with or without pre-functionalized surfaces.

The present invention may also find applications where the structure is used as filter, for example, in combination with nanoplasmonic sensing. The electrically conductive layer may be used as an electrode, both in this and other applications. For example, this may be used to measuring changes in the conductivity of the pore structure.

The present invention may find application within a number of technical fields, such as for instance for sensing protein interactions with surface immobilized target species, virology, cell analysis, DNA analysis, antibody-antigen analysis, drug discovery, diagnostic applications, and so on.

It should be noted that geometrical dimensions used in the above mentioned examples of manufacture are only used to give an indication of sizes and that these may be varied in a large range depending on used materials, process steps, and functionality as appreciated by the skilled person and should not be limiting to the present invention.

It should be noted that the word “comprising” does not exclude the presence of other elements or steps than those listed and the words “a” or “an” preceding an element do not exclude the presence of a plurality of such elements. It should further be noted that any reference signs do not limit the scope of the claims, and that several “means” or “units” may be represented by the same item of hardware.

The above mentioned and described embodiments are only given as examples and should not be limiting to the present invention. Other solutions, uses, objectives, and functions within the scope of the invention as claimed in the below described patent claims should be apparent for the person skilled in the art.

1. A nanoplasmonic device (201) comprising a membrane (104) with at least one layer of conducting material, wherein the membrane is perforated with a plurality of through going channels (110, 111, 211), and wherein the relative position of the channels are arranged so as to form a pattern with no long-range order.

2. The device according to claim 1, wherein the spatial length between nearest neighbour through going channels are of the order of 1 to 10 000 nanometres, preferably of the order 10 to 1 000 nanometers, and more preferably of the order 50 to 500 nanometers.

3. The device according to claim 1, wherein the through going channels have a diameter of the order 10 to 500 nanometers preferably of the order 25 to 250 nanometers, and more preferably 50 to 150 nanometers.

4. The device according to claim 1, wherein the electrically conducting layer comprise at least one of gold, silver, palladium, and platinum.

5. The device according to claim 1, wherein a thickness of the membrane is of the order of 1 to 1000 nanometers, preferably 5 to 500 nanometers, and more preferably 10 to 100 nanometers.

6. The device according to claim 4, wherein electrically conducting layer also comprise at least one of chrome, titanium, chrome oxide, titanium oxide, and tantalum oxide.

7. The device according to claim 1, wherein the membrane further comprises at least one mechanically stabilizing layer (101).

8. The device according to claim 7, wherein the mechanically stabilizing layer is one of an insulating layer or semi conducting layer.

9. A measurement system (300) for measuring molecular reactions, comprising:
at least one nanoplasmonic device (201) according to claim 1;
a fluid flow cell (212) arranged so as to provide contact by fluid in the fluid flow cell with the sensor consumable;
a system (202, 210) for determining optical properties of the sensor consumable;
a control and analysis system (302) in electrical connection with the system for determining optical properties.

10. A method of manufacturing a nanoplasmonic device (201), comprising the steps of:
forming a membrane (104);
forming a plurality of through going channels (110, 111, 211) in the membrane and wherein the relative position of the channels are arranged so as to form a pattern with no long-range order.

11. The method according to claim 10, wherein the steps of forming the membrane and the channels comprise the steps of:
depositing colloids (103) on a mechanically stabilizing layer (101, 102) with a spatial length in a range of 1 colloid per 1 to 10 000 nm;
evaporating an electrically conducting layer (104) on the mechanically stabilizing layer and the colloids;
removing the colloids forming holes in the electrically conducting layer;
coating the mechanically stabilizing layer and conducting layer with an insulating layer;
defining and removing a window structured portion (108) in a substrate back side and removing a windowed structure portion (109) of the mechanically stabilizing layer exposed after removal of the windowed structured portion of the substrate;
producing through going channels (110, 111, 211) through the mechanically stabilizing layer and the conduction layer forming nano sized pores with nanoplasmonic properties.

12. The method according to claim 11, wherein the step of depositing colloids comprises depositing the colloids in a homogenous short-range order.

13. The method according to claim 11, further comprising an initial step of depositing an insulating or semi conducting layer (101) on the substrate (102).

14. The method according to claim 11, wherein the mechanically stabilizing layer is one of a substrate layer (102) or a separate insulating or semi conducting layer (101).

15. A sensor consumable, comprising:
a nanoplasmonic device (201) according to claim 1; and
a holding structure arranged to be held by the measurement system according to claim 9.

16. A method of measuring molecular reactions with a nanoplasmonic device (201), comprising the steps of:
placing a nanoplasmonic device (201) with no long-range ordered through going channels (110, 111) in contact with a fluid flow cell (211);
providing a reactant to a fluid;
providing the fluid with the reactant to the fluid flow cell;
determining optical properties of the nanoplasmonic device over time;
relating changes of the optical properties to molecular reactions.
